(19) United States
(12) Patent Application Publication
(10) Pub. No.: US 2021/0077774 A1
(43) Pub. Date: Mar. 18, 2021

(54) DUAL LUMEN CANNULA WITH FLEXIBLE DISTAL END

(71) Applicant: CARDIACASSIST, INC., Pittsburgh, PA (US)

(72) Inventors: Patrick A. Kelly, North Huntingdon, PA (US); Robert G. Svitek, Freeport, PA (US); Jerry Stokes, Sarver, PA (US)

(73) Assignee: CARDIACASSIST, INC., Pittsburgh, PA (US)

(21) Appl. No.: 16/951,523

(22) Filed: Nov. 18, 2020

Related U.S. Application Data
(63) Continuation of application No. PCT/US2019/036987, filed on Jun. 13, 2019.
(60) Provisional application No. 62/684,858, filed on Jun. 14, 2018.

(51) Int. Cl. A61M 25/00; A61M 1/00; A61M 25/10

(52) U.S. Cl. CPC A61M 25/003; A61M 1/0058; A61M 25/0028; A61M 25/0052; A61M 2210/12; A61M 25/10; A61M 2025/0039; A61M 2205/0266; A61M 25/0074

(57) ABSTRACT

A dual lumen coaxial cannula assembly includes a first infusion tube having a first elongate body defining a first lumen therethrough, the first infusion tube having a proximal end, a distal end, and a sidewall extending circumferentially therebetween. A second drainage tube is co-axially aligned with the first infusion tube and has a second elongate body with a second lumen defined by a space between the first infusion tube and the second drainage tube. The second drainage tube has a proximal end, a distal end, and a sidewall extending circumferentially therebetween. A connector is attached to the proximal end of the first infusion tube and the proximal end on the second drainage tube. The cannula assembly further includes a distal tip connected to the distal end of the first elongate body and made of a material having a lower hardness than a hardness of the first elongate body.

### CROSS REFERENCE TO RELATED APPLICATION

This application is a continuation of International Application No. PCT/US2019/036987, filed Jun. 13, 2019, which claims priority to U.S. Provisional Application No. 62/684,858, filed Jun. 14, 2018, the disclosures of which are hereby incorporated in its entirety.

### BACKGROUND

### Field

The present disclosure generally relates to devices and methods for assisting a patient's heart with a cannula. More specifically, the present disclosure is related to a cannula assembly having a flexible tip to provide safe and efficient positioning in the patient's vasculature, as well as to methods for assisting a patient's heart with a cannula assembly.

### Description of the Related Art

Examples of existing cannula devices are described in U.S. Pat. Nos. 9,168,352, 9,782,534, and 10,279,101, the disclosures of which are hereby incorporated by reference in their entireties.

Traditional cannulae used for patient life support generally involve single lumen cannulae at multiple insertion sites, high volume circuits, and cannulae that are not capable of long-term use. Multiple insertion sites increase the risk of bleeding, vessel damage, and infection, as well as pain and discomfort to the patient. These cannulae are designed and built for short-term acute therapies. Additionally, traditional cannulae usually require access sites located in the patient's groin area near the right or left femoral veins.

Patients with severe right-sided circulatory and/or right-sided ventricular failure have significantly high mortality and morbidity caused by a multitude of factors in multiple patient populations. Historically, Right Ventricular Assist Devices (RVADs) and Left Ventricular Assist Devices (LVADs) have been adapted for use on surgical patients without a percutaneous or catheter lab option available. These surgical RVADs have been applied on patients with right inferior myocardial infarction, acute right-sided ischemic myocardial infarctions (with large left and right propagation), cardiogenic shock, LVAD-created right ventricular dysfunction, post-transplant right ventricular failure, and pulmonary hypertension. Acute myocardial infarction and cardiogenic shock have been treated with intra-aortic balloon pumps and maximal inotropic support, to which many patients become refractory. Surgically implanted LVADs can create a significant septal shift that leads to a dynamic change in the Starling curve that abruptly places patients into severe right ventricular failure. Patients can limit post-transplant survival bridged to transplant to/from an RVAD with severe right ventricular failure. Secondary pulmonary hypertension leads to an exacerbation of right ventricular failure in acute and chronic situations, which may be treated with RVADs.

Some conventional devices do not have the capability to reach the pulmonary artery (PA) from the internal jugular vein via a percutaneous insertion. Some traditional cannulae are inserted into the patient's heart through a direct access point in the patient's right or left femoral vein. Alternatively, traditional RVADs have a cannula either primarily placed in the PA or a graft sewn onto the PA, then a cannula inserted through the graft. The assembly can then be visualized in the PA via fluoroscopy and X-ray with the aid of distal markers in the cannula, verifying the proper orientation of the outflow to the patient. In these embodiments, the patient's torso length can limit the ability to access the PA via percutaneous insertion. If a cannula is not of a proper length, the interventional procedure may not unload the right ventricle, which leads to an increase of morbidity and mortality.

Furthermore, traditional venoarterial extracorporeal membrane oxygenation (VA ECMO) is the current standard of care used to treat right ventricular failure and respiratory failure percutaneously. A VA ECMO procedure draws blood from the right atrium and pumps it through an oxygenator and back into the arterial circulation via the femoral artery. VA ECMO bypasses the lungs and the heart completely. Therefore, residual blood is left stagnant in both the heart and lungs, potentially leading to thrombosis and an inadequately unloaded right ventricle. Additionally, the arterial cannulation can lead to problems including but not limited to bleeding, stroke, and infection.

Still further, some conventional devices are known to cause abrasion and other injury to the vessel walls, as well as discomfort to the patient due to the rigidity necessary to maneuver through the complex pathways of a patient's vasculature..

### SUMMARY

In view of the foregoing, there is a need for a dual lumen cannula with a single insertion point. There is an additional need for a dual lumen cannula that eliminates multiple access sites and reduces bleeding, vessel damage, and infection, as well as pain and discomfort to the patient. Furthermore, there exists a need for a dual lumen cannula that enables patients to be ambulatory with access sites provided in the neck area instead of the groin.

Embodiments of the present disclosure are directed to a dual lumen coaxial cannula assembly. The cannula assembly includes a first infusion tube having a first elongate body defining a first lumen therethrough, the first infusion tube having a proximal end, a distal end, and a sidewall extending circumferentially therebetween; a second drainage tube co-axially aligned with the first infusion tube and having a second elongate body with a second lumen defined by a space between the first infusion tube and the second drainage tube, the second drainage tube having a proximal end, a distal end, and a sidewall extending circumferentially therebetween; a connector attached to the proximal end of the first infusion tube and the proximal end of the second drainage tube; and a distal tip defining a distal lumen therethrough, the distal tip having a proximal end, a distal end, and a sidewall extending circumferentially therebetween, the distal tip connected to the distal end of the first elongate body. The distal tip is manufactured of a material having a lower hardness than a hardness of the first elongate body.

In some embodiments, the distal tip is connected to a distal end of the first elongate body along a plane perpendicular to a longitudinal axis of the first infusion tube.

In some embodiments, the distal tip and the distal end of the first elongate body are tapered to increase a surface area of a connection between the distal tip and the distal end of the first elongate body.

In some embodiments, the distal tip and the distal end of the first elongate body are roughened to increase a surface area of a connection between the distal tip and the distal end of the first elongate body.

In some embodiments, the sidewall of the first infusion tube defines a shoulder such that a portion of the sidewall of the first infusion tube extending distally from the shoulder has a lesser outer diameter than a portion of the sidewall of the first infusion tube extending proximally from the shoulder. The sidewall of the distal tip defines a bore adapted to receive the portion of the sidewall of the first infusion tube extending distally from the shoulder, such that a portion of the sidewall of the distal tip overlaps the portion of the sidewall of the first infusion tube extending distally from the shoulder.

In some embodiments, the cannula assembly further includes a basket disposed between the sidewall of the distal tip and the sidewall of the first infusion tube, the basket comprising a plurality of individual members arranged to prevent collapse of the distal lumen.

In some embodiments, the basket is comprised of a shape memory material defining a first profile at a first temperature and a second profile at a second temperature.

In some embodiments, the first profile is a straight tubular profile, the first temperature is a temperature at which the distal tip is connected to the distal end of the first elongate body during a manufacturing process of the dual lumen coaxial cannula assembly, the second profile is a tubular structure curving about an axis perpendicular to a longitudinal axis of the first elongate body, and the second temperature is a human body temperature. The basket transitions from the first profile to the second profile as a temperature of the dual lumen coaxial cannula assembly transitions from the first temperature to the second temperature.

In some embodiments, the second profile is adapted for positioning in a bend of a patient's pulmonary artery.

In some embodiments, a profile of the distal tip defines a tubular structure curving about an axis perpendicular to a longitudinal axis of the first elongate body.

In some embodiments, the profile of the distal tip is adapted for positioning in a bend of a patient's pulmonary artery.

In some embodiments, the distal end of the distal tip defines an end feature shaped to reduce the prevalence of edges of the distal tip in order to ease venous insertion of the dual lumen coaxial cannula assembly into the patient and reduce the risk of vessel wall injury.

In some embodiments, the end feature has a profile of at least one of a balloon, a hemisphere, an oval, and a parabola.

In some embodiments, the distal tip further includes a flap movable between a closed position preventing fluid flow out of the distal lumen and an opened position permitting fluid flow out of the distal lumen. In the closed position of the flap, fluid flow into the distal lumen creates backpressure forcing fluid out of one or more infusion apertures in the first elongate body.

In some embodiments, the flap is biased in the closed position and movable to the opened position by application of a force to a distal side of the flap.

Other embodiments of the present disclosure are directed to a method of inserting a cannula assembly into a patient's vasculature. The method includes inserting an introducer into the patient's jugular vein; maneuvering a distal end of the introducer through the patient's right atrium and right ventricle and into the patient's pulmonary artery, the introducer having a length such that a proximal end of the introducer extends out of the patient's body when the distal end of the introducer is positioned in the patient's pulmonary artery; sliding a central lumen of the cannula assembly over the introducer and maneuvering the cannula assembly along the introducer such that a first distal end of the cannula is positioned at least within proximity of the patient's pulmonary artery and such that a second distal end of the cannula is at least within proximity of the patient's right atrium; and removing the introducer from the patient through the central lumen of the cannula assembly. The cannula assembly includes a distal tip connected to the first distal end of the cannula assembly, wherein the distal tip is comprised of a material having a lower hardness than a hardness of the first distal end.

In some embodiments, as the central lumen of the cannula assembly is slid over the introducer, the introducer deflects a flap in the cannula assembly from a closed position for preventing fluid flow out of a distal end of the central lumen to an opened position. Removing the introducer allows the flap to deflect back to the closed position for preventing fluid flow out of a distal end of the central lumen.

In some embodiments, after removal of the introducer, the distal tip of the cannula assembly transitions from a first profile defining a straight tubular structure to a second profile defining a tubular structure curved about an axis perpendicular to a longitudinal axis of the cannula assembly.

In some embodiments, the transition from the first profile to the second profile is caused by a temperature change of a shape memory material forming at least a portion of the distal tip.

In some embodiments, the temperature change may include a change from a first temperature to a second temperature. The first temperature may be a temperature at which the distal tip is connected to the distal end of the first elongate body during a manufacturing process of the cannula assembly, and the second temperature may be a body temperature of the patient.

Further embodiments of the present disclosure are set forth in the following numbered clauses.

Clause 1. A dual lumen coaxial cannula assembly comprising: a first infusion tube having a first elongate body defining a first lumen therethrough, the first infusion tube having a proximal end, a distal end, and a sidewall extending circumferentially therebetween; a second drainage tube co-axially aligned with the first infusion tube and having a second elongate body with a second lumen defined by a space between the first infusion tube and the second drainage tube, the second drainage tube having a proximal end, a distal end, and a sidewall extending circumferentially therebetween; a connector attached to the proximal end of the first infusion tube and the proximal end on the second drainage tube; and a distal tip defining a distal lumen therethrough, the distal tip having a proximal end, a distal end, and a sidewall extending circumferentially therebetween, the distal tip connected to the distal end of the first elongate body, wherein the distal tip is comprised of a material having a lower hardness than a hardness of the first elongate body.

Clause 2. The dual lumen coaxial cannula assembly of clause 1, wherein the distal tip is connected to a distal end of the first elongate body along a plane perpendicular to a longitudinal axis of the first infusion tube.

Clause 3. The dual lumen coaxial cannula assembly of clause 1 or 2, wherein the distal tip and the distal end of the first elongate body are tapered to increase a surface area of a connection between the distal tip and the distal end of the first elongate body.

Clause 4. The dual lumen coaxial cannula assembly of any of clauses 1 to 3, wherein the distal tip and the distal end of the first elongate body are roughened to increase a surface area of a connection between the distal tip and the distal end of the first elongate body.

Clause 5. The dual lumen coaxial cannula assembly of any of clauses 1 to 4, wherein the sidewall of the first infusion tube defines a shoulder such that a portion of the sidewall of the first infusion tube extending distally from the shoulder has a smaller outer diameter than a portion of the sidewall of the first infusion tube extending proximally from the shoulder; and wherein the sidewall of the distal tip defines a bore adapted to receive the portion of the sidewall of the first infusion tube extending distally from the shoulder, such that a portion of the sidewall of the distal tip overlaps the portion of the sidewall of the first infusion tube extending distally from the shoulder.

Clause 6. The dual lumen coaxial cannula assembly of any of clauses 1 to 5, further comprising a basket disposed between the sidewall of the distal tip and the sidewall of the first infusion tube, the basket comprising a plurality of individual members arranged to prevent collapse of the distal lumen.

Clause 7. The dual lumen coaxial cannula assembly of any of clauses 1 to 6, wherein the basket is comprised of a shape memory material defining a first profile at a first temperature and a second profile at a second temperature.

Clause 8. The dual lumen coaxial cannula assembly of any of clauses 1 to 7, wherein the first profile is a straight tubular profile; wherein the first temperature is a temperature at which the distal tip is connected to the distal end of the first elongate body during a manufacturing process of the dual lumen coaxial cannula assembly; wherein the second profile is a tubular structure curving about an axis perpendicular to a longitudinal axis of the first elongate body; wherein the second temperature is a human body temperature; and wherein the basket transitions from the first profile to the second profile as a temperature of the dual lumen coaxial cannula assembly transitions from the first temperature to the second temperature.

Clause 9. The dual lumen coaxial cannula assembly of any of clauses 1 to 8, wherein the second profile is adapted for positioning in a bend of a patient's pulmonary artery.

Clause 10. The dual lumen coaxial cannula assembly of any of clauses 1 to 9, wherein a profile of the distal tip defines a tubular structure curving about an axis perpendicular to a longitudinal axis of the first elongate body.

Clause 11. The dual lumen coaxial cannula assembly of any of clauses 1 to 10, wherein the profile of the distal tip is adapted for positioning in a bend of a patient's pulmonary artery.

Clause 12. The dual lumen coaxial cannula assembly of any of clauses 1 to 11, wherein the distal end of the distal tip defines an end feature shaped to reduce the prevalence of edges of the distal tip in order to ease venous insertion of the dual lumen coaxial cannula assembly into the patient and reduce the risk of vessel wall injury.

Clause 13. The dual lumen coaxial cannula assembly of any of clauses 1 to 12, wherein the end feature has a profile of at least one of a balloon, a hemisphere, an oval, and a parabola.

Clause 14. The dual lumen coaxial cannula assembly of any of clauses 1 to 13, wherein the distal tip further comprises a flap movable between a closed position preventing fluid flow out of the distal lumen and an opened position permitting fluid flow out of the distal lumen; and wherein, in the closed position of the flap, fluid flow into the distal lumen creates backpressure forcing fluid out of one or more infusion apertures in the first elongate body.

Clause 15. The dual lumen coaxial cannula assembly of any of clauses 1 to 14, wherein the flap is biased in the closed position and movable to the opened position by application of a force to a distal side of the flap.

Clause 16. A method of inserting a cannula assembly into a patient's vasculature, the method comprising: inserting an introducer into the patient's jugular vein; maneuvering a distal end of the introducer through the patient's right atrium and right ventricle and into the patient's pulmonary artery, the introducer having a length such that a proximal end of the introducer extends out of the patient's body when the distal end of the introducer is positioned in the patient's pulmonary artery; sliding a central lumen of the cannula assembly over the introducer and maneuvering the cannula assembly along the introducer such that a first distal end of the cannula assembly is positioned at least within proximity of the patient's pulmonary artery and such that a second distal end of the cannula assembly is at least within proximity of the patient's right atrium; and removing the introducer from the patient through the central lumen of the cannula assembly, wherein the cannula assembly comprises a distal tip connected to the first distal end of the cannula assembly, wherein the distal tip is comprised of a material having a lower hardness than a hardness of the first distal end.

Clause 17. The method of clause 16, wherein, as the central lumen of the cannula assembly is slid over the introducer, the introducer deflects a flap in the cannula assembly from a closed position for preventing fluid flow out of a distal end of the central lumen to an opened position; and wherein removing the introducer allows the flap to deflect back to the closed position for preventing fluid flow out of a distal end of the central lumen.

Clause 18. The method of clause 16 or 17, wherein, after removal of the introducer, the distal tip of the cannula assembly transitions from a first profile defining a straight tubular structure to a second profile defining a tubular structure curved about an axis perpendicular to a longitudinal axis of the cannula assembly.

Clause 19. The method of any of clauses 16 to 18, wherein the transition from the first profile to the second profile is caused by a temperature change of a shape memory material forming at least a portion of the distal tip.

Clause 20. The method of any of clauses 16 to 19, wherein the temperature change comprises a change from a first temperature to a second temperature, wherein the first temperature is a temperature at which the distal tip is connected to the distal end of the first elongate body during a manufacturing process of the cannula assembly, and wherein the second temperature is a body temperature of the patient.

Further details and advantages of the present disclosure will be understood from the following detailed description read in conjunction with the accompanying drawings.

### BRIEF DESCRIPTION OF THE DRAWINGS

FIG. 1 is a top view of one embodiment of a coaxial cannula shown with a connector.

FIG. 2 is a side view of the coaxial cannula shown in FIG. 1.

FIG. 3 is a top view of one embodiment of an infusion cannula.

FIG. 4A is a cross-sectional view of detail A shown in FIG. 3, according to an embodiment of the coaxial cannula.

FIG. 4B is a cross-sectional view of detail A shown in FIG. 3, according to another embodiment of the coaxial cannula.

FIG. 4C is a cross-sectional view of detail A shown in FIG. 3, according to another embodiment of the coaxial cannula.

FIG. 4D is a cross-sectional view of detail A shown in FIG. 3, according to another embodiment of the coaxial cannula.

FIG. 4E is a cross-sectional view of detail A shown in FIG. 3, according to another embodiment of the coaxial cannula.

FIG. 4F is an exploded perspective view of detail A shown in FIG. 3, according to another embodiment of the coaxial cannula.

FIG. 4G is a cross-sectional view of FIG. 4F.

FIG. 4H is a cross-sectional view of detail A shown in FIG. 3, according to another embodiment of the coaxial cannula.

FIG. 5 is a top view of one embodiment of a drainage cannula.

FIG. 6 is a cross-sectional view of detail B in FIG. 5.

FIG. 7 is a top cross-sectional view of the coaxial cannula taken along line A-A in FIG. 2.

FIG. 8 is a cross-sectional view of detail C in FIG. 7, illustrating a transition portion at a distal end of a drainage cannula of the coaxial cannula.

FIG. 9 is a perspective view of a connector shown coupled to a coaxial cannula according to another embodiment.

FIG. 10 is a rear perspective view of the coaxial cannula shown in FIG. 9.

FIG. 11 is a schematic view of one embodiment of a coaxial cannula positioned in the superior vena cava of a body of a patient.

FIG. 12 is a schematic view of one embodiment of a coaxial cannula positioned inside a patient's heart.

FIG. 13 is a schematic view of the coaxial cannula of any of FIGS. 4F-H positioned inside a patient's heart.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects. Hence, specific dimensions and other physical characteristics related to the embodiments or aspects disclosed herein are not to be considered as limiting.

As used herein, the term “at least one of” is synonymous with “one or more of”. For example, the phrase “at least one of A, B, and C” means any one of A, B, and C, or any combination of any two or more of A, B, and C. For example, “at least one of A, B, and C” includes one or more of A alone; or one or more B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of”. For example, the phrase “at least two of D, E, and F” means any combination of any two or more of D, E, and F. For example, “at least two of D, E, and F” includes one or more of D and one or more of E; or one or more of D and one or more of F; or one or more of E and one or more of F; or one or more of all of D, E, and F.

When used in relation to a cannula, catheter, or other device inserted into a patient, the term “proximal” refers to a portion of such device farther from the end of the device inserted into the patient. When used in relation to a cannula, catheter, or other device inserted into a patient, the term “distal” refers to a portion of such device nearer to the end of the device inserted into the patient.

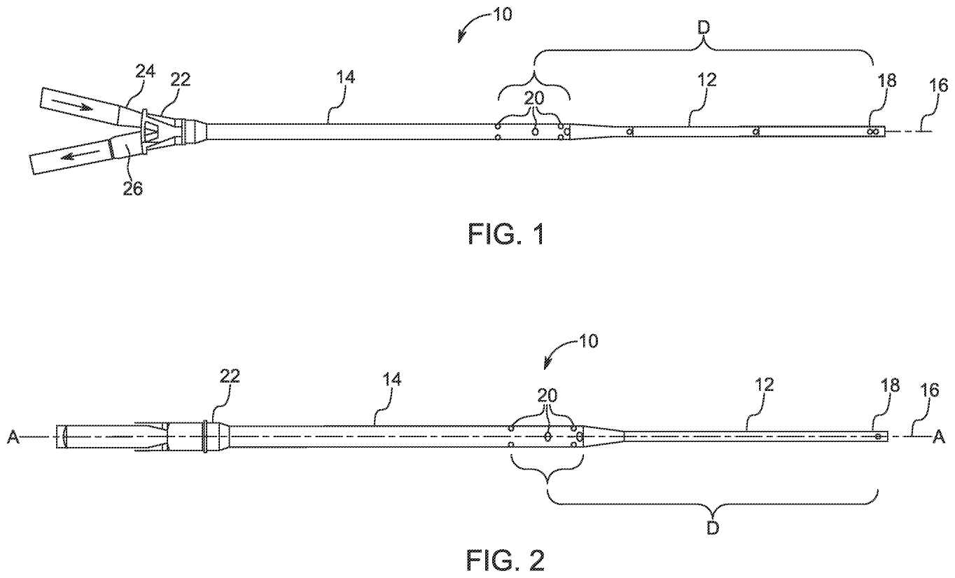

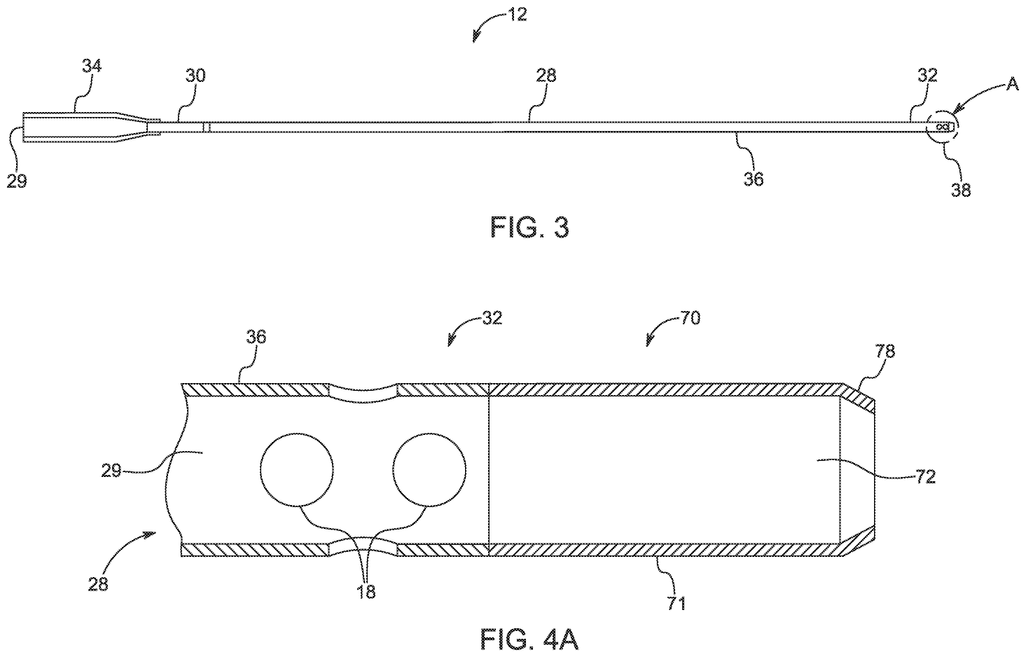

Referring to the drawings, in which like reference characters refer to like parts throughout the several views thereof, various embodiments of a coaxial, dual lumen cannula 10 (hereinafter referred to as “coaxial cannula 10”) are shown. With initial reference to FIGS. 1-2, the assembled coaxial cannula 10, according to one embodiment, generally includes a first infusion tube 12 having a first length and a second drainage tube 14 having a second length. The first length of the first infusion tube 12 is greater than the second length of the second drainage tube 14.

The first infusion tube 12 is disposed within the second drainage tube 14 in a coaxial arrangement centered about a central axis 16. Each of the first infusion tube 12 and the second drainage tube 14 has a first circumference defining a first lumen and a second circumference defining a second lumen, respectively. The first circumference of the first infusion tube 12 is smaller than the second circumference of the second drainage tube 14 such that the first infusion tube 12 may be placed within the second lumen of the second drainage tube 14. One or both of the first infusion tube 12 and the second drainage tube 14 may be manufactured from a medical-grade material such as polyurethane. Alternatively, the tubes may be made from PVC or silicone, and may be dip molded, extruded, co-molded, or made using any other suitable manufacturing technique.

The coaxial cannula 10 has sufficient placement flexibility adapted for placement of the coaxial cannula 10 within a patient's body. A vascular insertion site is provided at the internal jugular vein on the patient's neck area or other suitable venous location. The coaxial cannula 10 is adapted for placement above or below the right atrium of the patient's heart. The coaxial cannula 10 may be used with an introducer, such as a guide wire, to guide the placement of the coaxial cannula 10 as it is inserted within the patient's body.

With continuing reference to FIGS. 1 and 2, the coaxial cannula 10 is designed to withdraw blood directly from the patient's heart and return blood back into the patient's heart. The function of the first infusion tube 12 is to deliver blood into the bloodstream of the patient, while the function of the second drainage tube 14 is to drain the blood from the patient's bloodstream, as will be described hereafter.

A plurality of infusion apertures 18 is provided at a distal end of the first infusion tube 12. The plurality of infusion apertures 18 is desirably arranged in a circular pattern extending around the outer circumference of the first infusion tube 12. In some embodiments, the plurality of infusion apertures 18 may be disposed in multiple groups provided at various sites on the first infusion tube 12. Similarly, the second drainage tube 14 includes a plurality of drainage apertures 20 provided at a distal end of the second drainage tube 14. The plurality of drainage apertures 20 is desirably arranged in a circular pattern extending around the outer circumference of the second drainage tube 14. In alternative embodiments, the plurality of drainage apertures 20 may be arranged in groups disposed at various sites along the length of the second drainage tube 14. The infusion apertures 18 are separated from the drainage apertures 20 by a distance D. In different embodiments of the coaxial cannula 10, the separation of infusion apertures 18 from drainage apertures 20 determines the amount of mixing of oxygenated blood and unoxygenated blood. This distance may vary based on the age and size of the patient, as well as the desired flow rates during the medical procedure where the coaxial cannula 10 is used. For example, a coaxial cannula 10 having a specific overall length and diameter, along with a desired pattern and distance between the infusion apertures 18 and the drainage apertures 20, may be selected based on age and/or size of the patient.

With continuing reference to FIGS. 1 and 2, a connector 22 is provided at the proximal end of the coaxial cannula 10. The connector 22 includes an inlet portion 24 in fluid communication with the first infusion tube 12 to transfer blood from a blood pump (not shown) to the first infusion tube 12. An outlet portion 26 of the connector 22 is in fluid communication with the second drainage tube 14 to transfer blood from the second drainage tube 14 to the blood pump. The outlet portion 26 and the inlet portion 24 of the connector 22 are arranged such that the fluid pathways leading from the second drainage tube 14 and to the first infusion tube 12 transition from a coaxial arrangement at a distal end of the connector 22 to an axially-offset arrangement at a proximal end of the connector 22. Details of the connector 22 construction will be discussed in greater detail below.

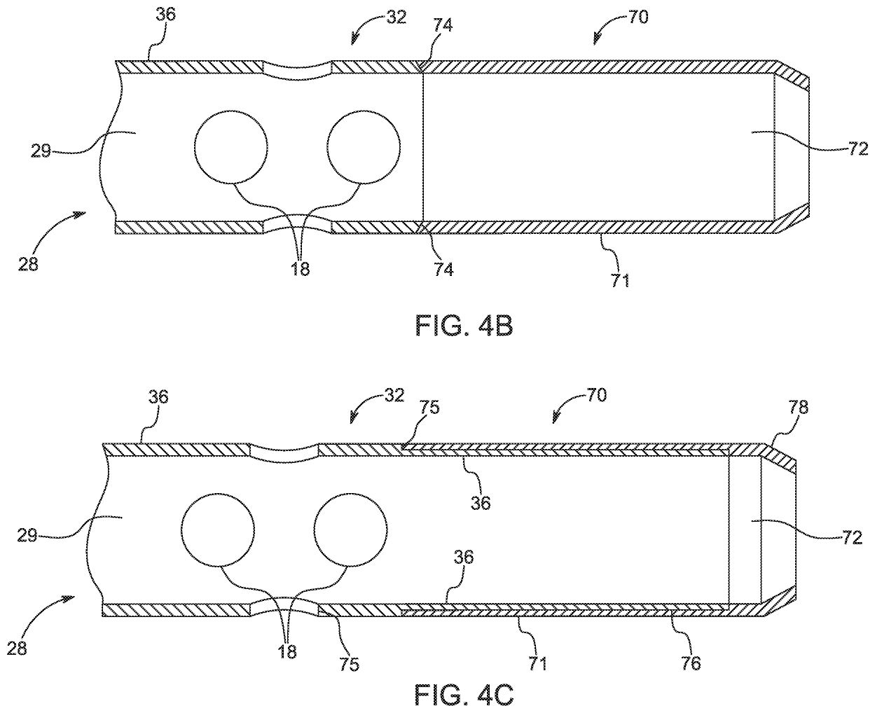

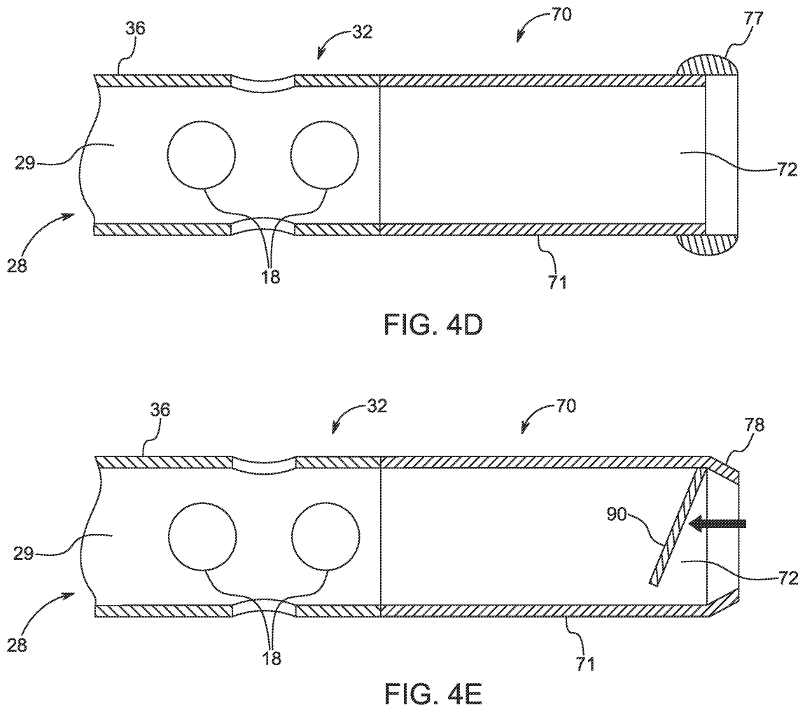

With reference to FIGS. 3-4G, and with continuing reference to FIGS. 1 and 2, the first infusion tube 12 of the coaxial cannula 10 is illustrated. The first infusion tube 12 has a first elongate body 28 having a working length of, for example, around 21 cm. The first elongate body 28 of the first infusion tube 12 is substantially cylindrical and extends from a first proximal end 30 to a first distal end 32. The first elongate body 28 includes a first lumen 29 extending throughout the entire length of the first infusion tube 12. The first proximal end 30 includes a first connector portion 34 for coupling the first infusion tube 12 to the inlet portion 24 of the connector 22. The first elongate body 28 of the first infusion tube 12 has a hollow structure defined by a first sidewall 36 extending circumferentially about the first elongate body 28. The first sidewall 36 has a substantially constant thickness throughout the length of the first elongate body 28. The first distal end 32 of the first elongate body 28 may be attached to a pliable distal tip 70 having a hollow structure defined by a tip sidewall 71. The distal tip 70 defines a tip lumen 72 in fluid communication with the first lumen 29 of the first elongate body 28 and allowing fluid flow between the tip lumen 72 and the first lumen 29 of the first elongate body 28. A distal end of the distal tip 70 may include a first tapering section 78 for enabling easier insertion of the first infusion tube 12 into the patient's body.

The distal tip 70 may be made from a medical grade material such as polyurethane, or another material suitable for bonding to the material of the first elongate body 28. In some embodiments, the distal tip 70 may be manufactured from a material having a Shore durometer hardness of less than or equal to 85 A to prevent injury and/or discomfort to the patient when the cannula 10 is placed into the patient. In some embodiments, the distal tip 70 may have a Shore durometer hardness of at least 5 A less than the Shore durometer hardness of the first elongate body 28.

With reference to FIGS. 4A-4H, various embodiments of the first distal end 32 of the first elongate body 28 and the distal tip 70 are shown. In one embodiment shown in FIG. 4A, the first sidewall 36 of the first elongate body 28 and the tip sidewall 71 of the distal tip 70 abut one another on a plane substantially perpendicular to the longitudinal axis of the first elongate body 28. The distal tip 70 may be fused to the first elongate body 28 via a reflow process utilizing heated glass molds or a heat-shrinkable material such as polyethylene terephthalate (PET). Alternatively, the distal tip 70 may be fused to the first elongate body 28 via radiofrequency welding. One or both of the distal tip 70 and the first elongate body 28 may be subjected to a buffing operation prior to joining in order to increase surface area of the connection between the distal tip 70 and the first elongate body 28, thereby increasing resistance to tensile separation of the distal tip 70 and the first elongate body 28. The embodiment shown in FIG. 4B is substantially identical to the embodiment shown in FIG. 4A, except that the distal tip 70 includes a connecting taper 74 interacting with a respective taper of the first elongate body 28 to further increase the surface area of the connection between the distal tip 70 and the first elongate body 28. The respective tapers of the distal tip 70 and the first elongate body 28 also assist in aligning the tip lumen 72 and the first lumen 29 of the first elongate body 28 during the fusing of the distal tip 70 and the first elongate body 28.

In one embodiment shown in FIG. 4C, the first elongate body 28 may include a shoulder 75 reducing the outer diameter of the first sidewall 36. The shoulder 75 may be formed by a grinding operation after the first elongate body 28 is molded or otherwise formed. In some embodiments, the distance from the shoulder 75 to the first distal end 32 of the first intrusion tube 12 may be approximately 5 centimeters. The thickness of the first sidewall 36 may be reduced by at least 50% at the shoulder 75. In some embodiments, the first sidewall 36 may have the same outer diameter from the shoulder 75 to the first distal end 32. In other embodiments, the outer diameter of the first sidewall 36 may taper from the shoulder 75 to the first distal end 32. The distal tip 70 has a bore 76 sized to slide over and interface with the reduced outer diameter of the first sidewall 36 and abut the shoulder 75, forming a lap joint between the distal tip 70 and the first elongate body 28. The interfacing external surface of the first sidewall 36 and the internal surface of the bore 76 of the distal tip 70 may be joined via a reflow process or radiofrequency welding as discussed with reference to the embodiment of FIG. 4A.

In one embodiment shown in FIG. 4D, the distal tip 70 may include an end feature such as a balloon 77 to ease insertion of the coaxial cannula 10 into the patient and further reduce patient discomfort and risk of vessel wall injury by eliminating sharp edges of the distal tip 70. Profiles of end features other than the balloon 77 which eliminate or mitigate sharp edges of the distal tip 70 may be appreciated by one skilled in the art. For example, the end feature may have a hemispherical, ovaline, teardrop, or parabolic profile.

In one embodiment shown in FIG. 4E, the distal tip 70 may include a flap 90 closable by fluid pressure to prevent fluid flow out of the tip lumen 72. Fluid flowing from the proximal end of the first elongate body 28 to the distal end of the first elongate body 28 applies pressure to a proximal side of the flap 90, biasing the flap 90 to a closed position and creating back pressure against fluid flow out of the infusion apertures 18. The flap 90 may be deflected to an opened position by application of a force to the distal side of the flap 90. For example, during insertion of the coaxial cannula 10 into the patient, the flap 90 may be moved to the opened position by the introducer as the coaxial cannula 10 is slid over the distal end of the introducer.

In one embodiment shown in FIGS. 4F-4G, a basket 80 may be disposed between the distal tip 70 and the first sidewall 36 to resist collapse of the tip lumen 72 due to forces on the tip sidewall 71. The basket 80 includes a plurality of individual members 81 extending over the outer surface of the first sidewall 36 to provide hoop strength to the first sidewall 36. The arrangement of the individual members 81 may form any design balancing improved hoop strength with pliability. In general, covering a greater area of the outer surface of the first sidewall 36 with the individual members 81 results in greater hoop strength but less pliability, and vice versa. However, one skilled in the art may appreciate and utilize various geometric arrangements of the individual members 81 that optimize hoop strength and pliability. Suitable materials for manufacturing the basket 80 include biocompatible metal or metal alloys such as stainless steel or nickel titanium alloy (nitinol). The individual members 81 may be formed via chemical or laser etching or other suitable processes. The basket 80 may be joined to the first sidewall 36 via an adhesive, such as biocompatible or medical-grade Loctite®, prior to joining the distal tip 70 to the first sidewall 36. The distal tip 70, which in this embodiment is designed such that the bore 76 fits over the basket 80, may then be joined to the sidewall 36 via a reflow process or radiofrequency welding as discussed with reference to the embodiments of FIGS. 4A and 4C.

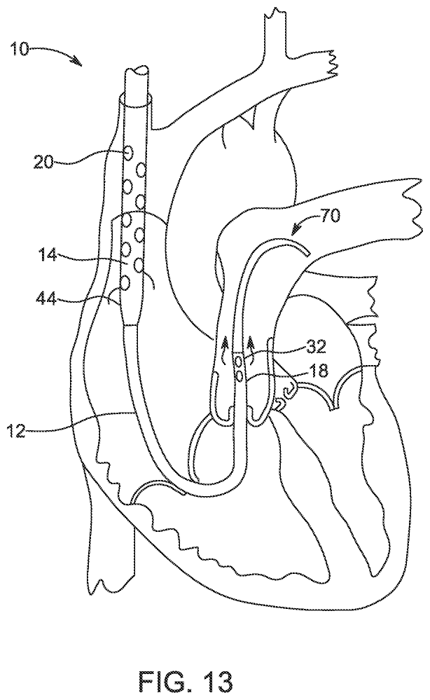

In some embodiments, the basket 80 may be manufactured of a shape memory material, such as nitinol, such that the basket 80 defines a first profile at a first temperature and a second profile at a second temperature. For example, the first profile of the basket 80 may be a substantially straight tubular structure as shown in FIG. 4F. The first profile may be exhibited at a manufacturing temperature at which the basket 80 and the distal tip 70 are joined to the first elongate body 28. The second profile of the basket 80 may be a curved tubular structure or pigtail, as shown in FIG. 13. Specifically, the second profile may be a tubular structure curved about an axis perpendicular to a longitudinal axis of the cannula assembly 10. The second profile may be exhibited at human body temperature, particular the body temperature within the pulmonary artery, for example 98.6° Fahrenheit. In such an embodiment, the straight first profile of the basket 80 facilitates assembly of the first infusion tube 12. As the first infusion tube 12 is inserted into the patient, the basket 80 and the distal tip 70 adhered thereto transitions to the curved second profile to facilitate placement of the distal tip 70 in the patient's pulmonary artery.

In one embodiment shown in FIG. 4H, the distal tip 70 may be pre-formed into a curved or pigtail profile. The distal tip 70 may be joined to the first elongate body 28 in any suitable manner described with reference to FIGS. 4A-4G. As noted with reference to FIG. 13, the curved or pigtail profile facilitates placement and retention of the distal tip 70 in the patient's pulmonary artery. Additionally, the distal tip 70 of the embodiment of FIG. 13 may be made of a particularly soft material relative to the first infusion tube 12 to prevent damage to the arterial walls of the patient. The tip lumen 72 of the distal tip 70 may be closed in some embodiments where the distal tip 70 is used only for retention in the pulmonary artery and not to deliver blood to the pulmonary artery.

With continued reference to FIGS. 4A-4H, the plurality of infusion apertures 18 is provided at the first distal end 32 of the first infusion tube 12. The plurality of infusion apertures 18 extends circumferentially around the first distal end 32. Each infusion aperture 18 has a diameter of, for example, about 1 mm. The plurality of infusion apertures 18 may be arranged in an alternating pattern of axially offset rows of infusion apertures 18 arranged around the circumference of the first infusion tube 12. Each of the plurality of infusion apertures 18 extends through the thickness of the first sidewall 36. The infusion apertures 18 illustrated in FIGS. 3 and 4 extend through the first sidewall 36 in a direction perpendicular to a longitudinal axis of the first elongate body 28. Alternatively, the plurality of infusion apertures 18 may extend through the thickness of the first sidewall 36 in an angled manner with respect to the longitudinal axis of the first elongate body 28. For example, the plurality of infusion apertures 18 may be arranged at an acute or obtuse angle with respect to a cross-sectional plane of the first infusion tube 12 extending perpendicular to the longitudinal axis of the first elongate body 28. In some embodiments, a wire mesh basket is provided inside the first lumen 29 of the first infusion tube 12 at a location surrounding the infusion apertures 18. The wire mesh basket supports and prevents the first sidewall 36 from collapsing due to being weakened by the formation of the plurality of infusion apertures 18. In one embodiment, one or more sensors (not shown) may be provided at the first distal end 32 of the first infusion tube 12. The sensor(s) may be adapted for measuring, for example, local blood pressure and/or oxygen concentration.

The total cross-sectional area of the plurality of infusion apertures 18 is desirably approximately equal to or greater than the cross-sectional area of the first lumen 29. If the cross-sectional area of the plurality of infusion apertures 18 is less than the cross-sectional area of the first lumen 29, an undesirable pressure drop may occur. This pressure drop reduces the flow throughput within the first lumen 29 and impairs the efficiency of the first infusion tube 12. Desirably, the total cross-sectional area of the plurality of infusion apertures 18 exceeds the cross-sectional area of the first lumen 29 such that if one or more of the infusion apertures 18 becomes clogged, the total cross-sectional area of the remaining infusion apertures 18 is equal to or greater than the cross-sectional area of the first lumen 29. In this manner, the blood flow through the first lumen 29 is maximized even if one or more of the infusion apertures 18 becomes clogged.

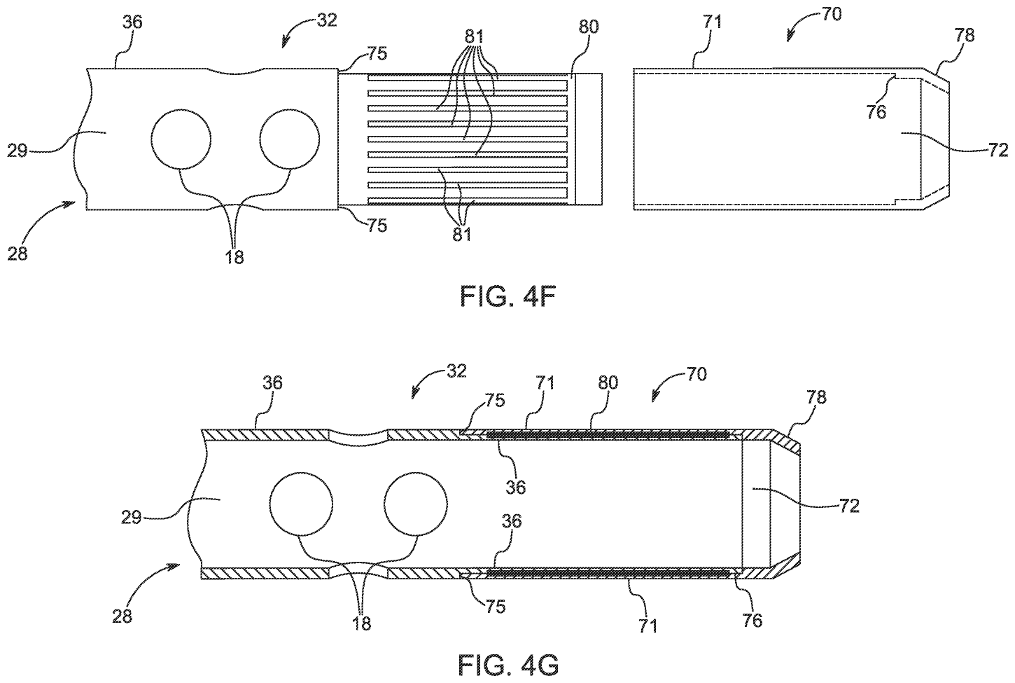

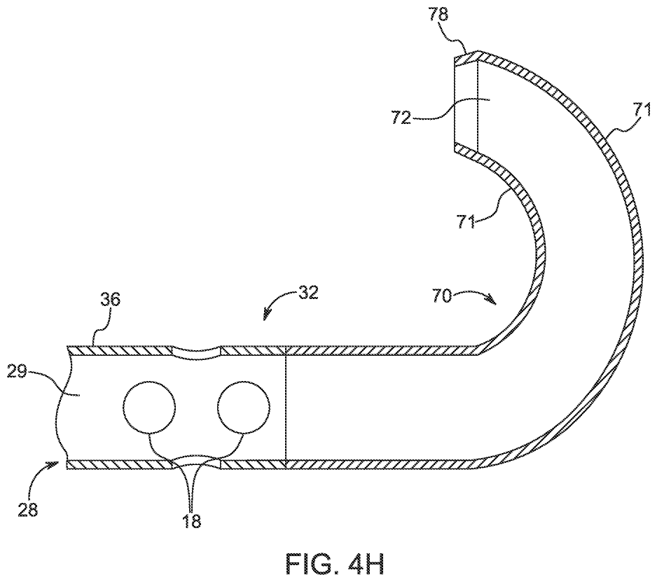

With reference to FIGS. 5-6, and with continuing reference to FIGS. 1 and 2, the second drainage tube 14 is illustrated separately from the coaxial cannula 10. The second drainage tube 14 has a second elongate body 40 having a working length of, for example, around 12 cm. The second elongate body 40 of the second drainage tube 14 is substantially cylindrical and extends from a second proximal end 42 to a second distal end 44. The second elongate body 40 includes a second lumen 46 extending throughout the entire length of the second drainage tube 14. The second proximal end 42 includes a second connector portion 47 for coupling the second drainage tube 14 to the outlet portion 26 of the connector 22. The second elongate body 40 of the second drainage tube 14 has a hollow structure defined by a second sidewall 48 extending circumferentially about the second elongate body 40. The second sidewall 48 has a substantially constant thickness throughout the length of the second elongate body 40, with a second tapering section 50 at the second distal end 44 of the second elongate body 40. At the second proximal end 42 of the second elongate body 40, the second sidewall 48 gradually increases in thickness before transitioning into the second connector portion 47. The second tapering section 50 located at the second distal end 44 has a thinner second sidewall 48 but retains the internal diameter of the second lumen 46. The second tapering section 50 enables easier insertion of the second drainage tube 14 into the patient's body.

With specific reference to FIG. 6, the second distal end 44 of the second drainage tube 14 is shown. The plurality of drainage apertures 20 is provided at the second distal end 44 of the second drainage tube 14. The plurality of drainage apertures 20 extends circumferentially around the second distal end 44. Each drainage aperture 20 has a diameter of, for example, about 1.5 mm. The plurality of drainage apertures 20 may be arranged in an alternating pattern of axially offset rows around the circumference of the second drainage tube 14. Each of the plurality of drainage apertures 20 extends through the thickness of the second sidewall 48. The drainage apertures illustrated in FIGS. 5 and 6 extend through the second sidewall 48 in a direction perpendicular to a longitudinal axis of the second elongate body 40. Alternatively, the plurality of drainage apertures 20 may extend through the thickness of the second sidewall 48 in an angled manner with respect to the longitudinal axis of the second elongate body 40. For example, the plurality of drainage apertures 20 may be arranged at an acute or obtuse angle with respect to a cross-sectional plane of the second drainage tube 14 extending perpendicular to the longitudinal axis of the second elongate body 40. In some embodiments, a wire mesh basket (not shown in FIG. 6) is provided inside the second lumen 46 of the second drainage tube 14 at a location surrounding the drainage apertures 20. The wire mesh basket supports and prevents the second sidewall 48 from collapsing due to being weakened by the formation of the plurality of drainage apertures 20. In one embodiment, one or more sensors (not shown) may be provided at the second distal end 44 of the second drainage tube 14. The sensor(s) may be adapted for measuring, for example, local blood pressure and/or oxygen concentration.

The total cross-sectional area of the plurality of drainage apertures 20 is desirably approximately equal to or greater than the cross-sectional area of the second lumen 46. If the cross-sectional area of the plurality of drainage apertures 20 is less than the cross-sectional area of the second lumen 46, an undesirable pressure drop within the second drainage tube 14 may occur. This pressure drop reduces the flow throughput within the second lumen 46 and impairs the efficiency of the second drainage tube 14. Desirably, the total cross-sectional area of the plurality of drainage apertures 20 exceeds the cross-sectional area of the second lumen 46 such that if one or more drainage apertures 20 becomes clogged, the total cross-sectional area of the remaining drainage apertures 20 is equal to or greater than the cross-sectional area of the second lumen 46. In this manner, the blood flow through the second lumen 46 is maximized even if one or more of the drainage apertures 20 becomes clogged.

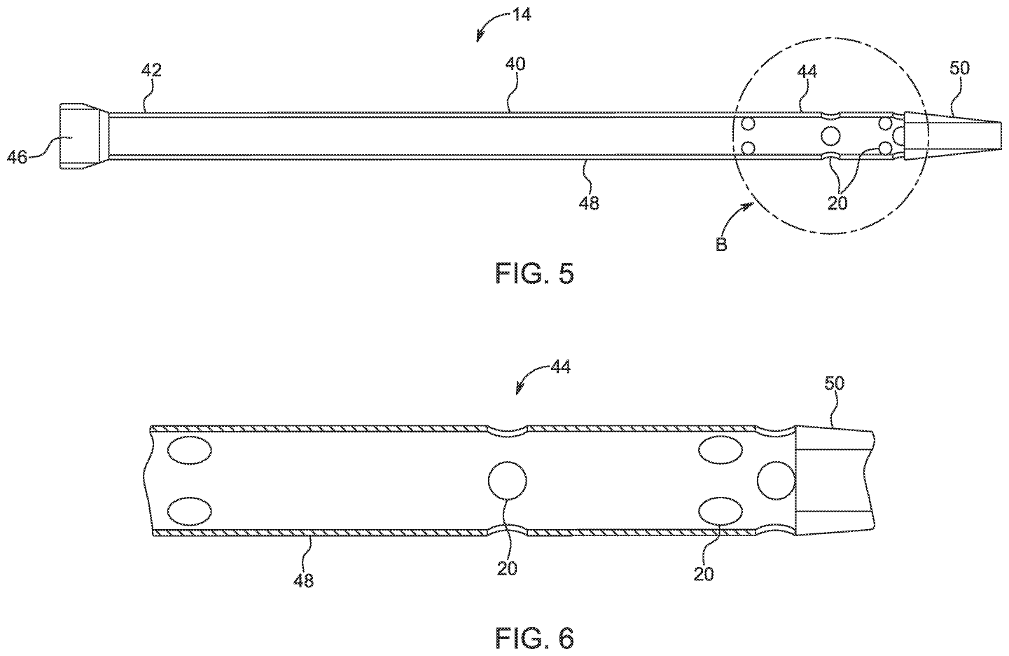

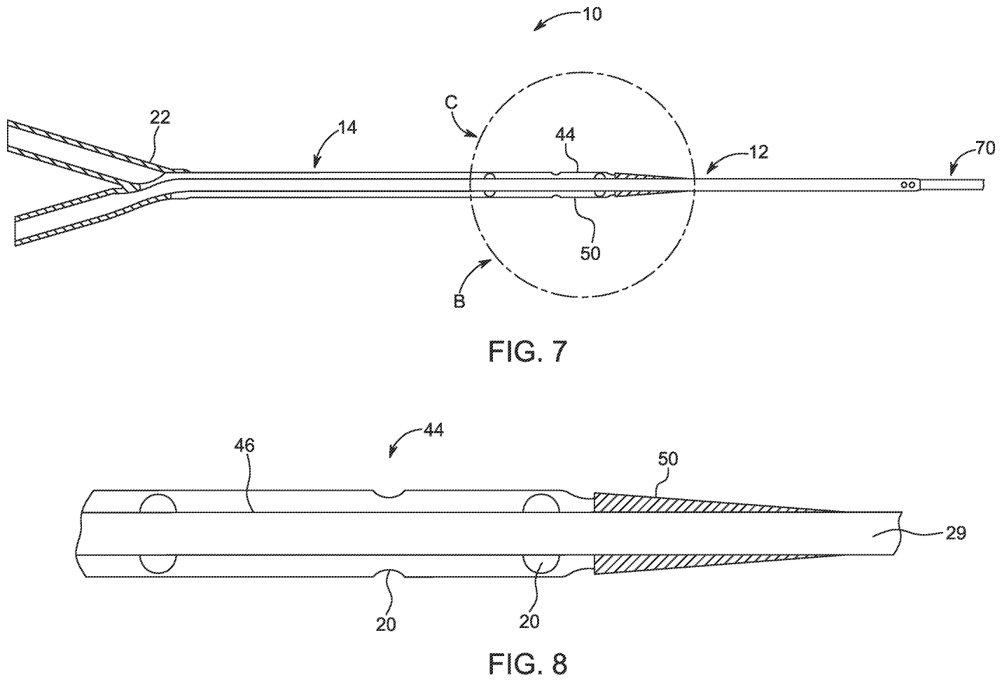

With reference to FIG. 7, the coaxial cannula 10 shown in FIGS. 1 and 2 is illustrated in cross section. The second distal end 44 of the second drainage tube 14 is fixedly attached to a mid portion of the first infusion tube 12 along the length of a second tapering section 50, as shown in FIG. 8. The first infusion tube 12 and the second drainage tube 14 are coupled to the connector 22 in such manner that the first infusion tube 12 and the second drainage tube 14 cross inside the connector 22 body without being connected to each other.

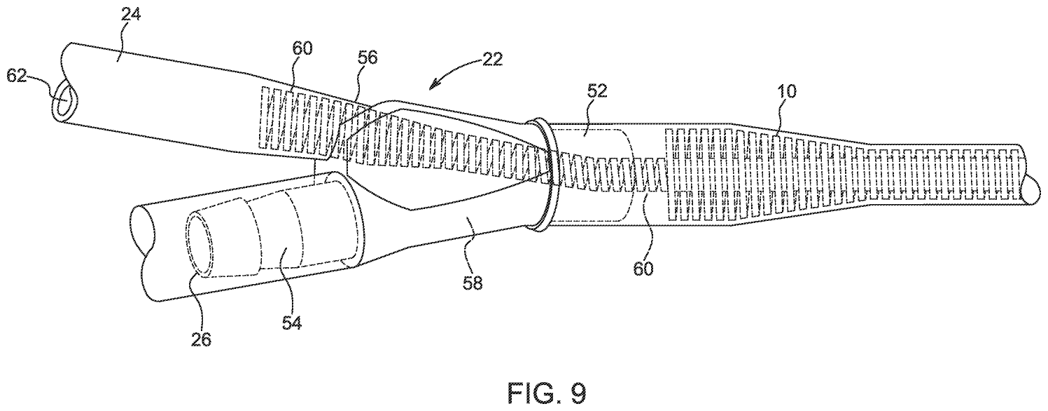

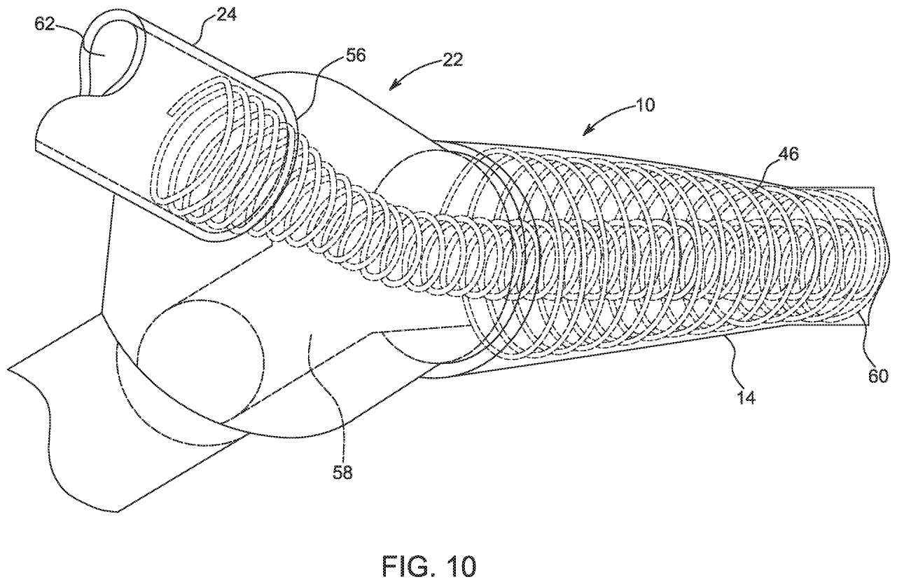

With reference to FIGS. 9 and 10, a detailed view of the connector 22 is shown coupled to a coaxial cannula 10 according to another embodiment. FIG. 9 illustrates the connector 22 showing the fluid pathways extending through the interior of the connector 22. The connector includes a distal aperture 52 at a distal end of the connector 22 for connecting to the proximal end of the coaxial cannula 10. The proximal end of the connector 22 has the inlet portion 24 and the outlet portion 26 in fluid communication with the distal aperture 52. The outlet portion 26 may include a barbed fitting 54 for connecting a drainage connection 57 that extends to a blood pump. The inlet portion 24 includes an inner tube 56 that extends through the interior of the connector 22 and connects with the first infusion tube 12. The inner tube 56 may extend beyond the distal aperture 52 for connecting with the first infusion tube 12. A drainage opening 58 connects the second drainage tube 14 with the outlet portion 26 of the connector 22. The drainage opening 58 is coextensive with the inner tube 56 along the length of the body portion of the connector 22. The inner tube 56 may be reinforced with a metal or plastic coil 60 that extends in a helical manner along the length of the inner tube 56 to minimize kinking and/or collapse of the first infusion tube 12.

With continuing reference to FIGS. 9 and 10, the inner tube 56 passes through the connector 22 to provide a smooth, seamless transition from a single distal aperture 52 to the branched arrangement of the inlet portion 24 and the outlet portion 26. The transition is desirably void of any joints, welds, and/or other connections that can create irregularities in the flow of blood and can damage blood cells.

In use, the proximal end of the coaxial cannula 10 is connected to the distal aperture 52 of the connector 22. The inner tube 56 receives blood from a supply line 62 and sends it through the lumen of the inner tube 56 to the first infusion tube 12. As the diameter of the inner tube 56 is smaller than the diameter of the drainage opening 58, the inner tube 56 extends through the interior of the connector 22, thus allowing the inner tube 56 to be continuous throughout the length of the connector 22. Depending on the application, the inner tube 56 may or may not include structural reinforcement in the form of the coil 60. In embodiments in which the inner tube 56 is reinforced with the coil 60, the inner tube 56 is stronger and less susceptible to kinking or collapse.

The connector 22 may be made from polycarbonate as an example, but could also be made from PVC, acrylic, or polyurethane. The connector 22 may be constructed using one or more manufacturing techniques including injection molding, machining, or dip forming. One of ordinary skill in the art will understand that a variety of other manufacturing techniques may be used for constructing the connector 22 without departing from the intended scope of the invention.

With continued reference to FIGS. 9 and 10, one or both of the first elongate body 28 of the first infusion tube 12 and the second elongate body 40 of the second drainage tube 14 includes a helical coil 60 extending through the length thereof. The helical coil 60 may be disposed along the interior surface of the first lumen 29 and the second lumen 46. Alternatively, the helical coil 60 may be disposed within the first sidewall 36 and the second sidewall 48. The helical coil 60 may be manufactured from medical-grade metal or plastic.

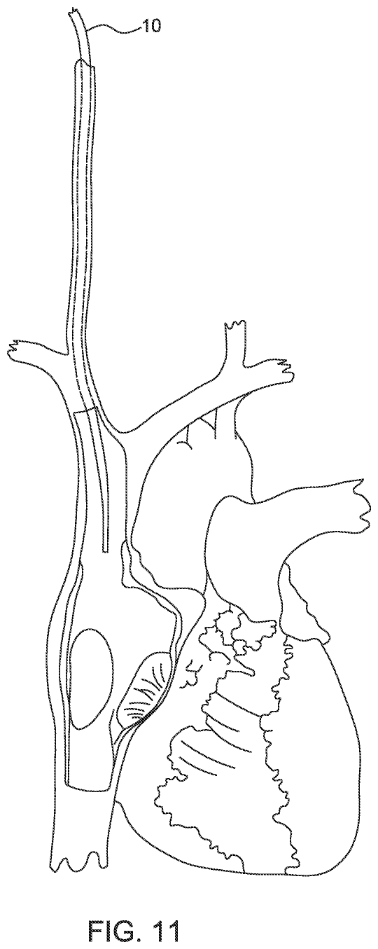

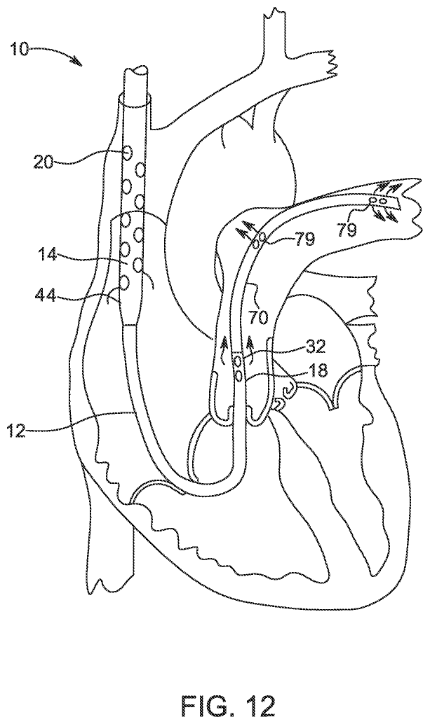

Having described several non-limiting embodiments of the coaxial cannula 10 and the connector 22, an exemplary and non-limiting method for supporting the right heart of a patient using the coaxial cannula 10 and the connector 22 will now be described with reference to FIG. 11. In use, the coaxial cannula 10 is inserted into the pulmonary artery (PA) in a percutaneous procedure. The coaxial cannula 10 withdraws blood from the patient's heart and delivers blood back to the patient. Initially, a percutaneous entry needle (not shown) is used to access the patient's internal jugular vein (IJV). An introducer, such as a guidewire, is then inserted through the needle until the tip of the introducer is positioned in the upper portion of the inferior vena cava/right atrium (IVC/RA) junction. The needle can then be removed and a pulmonary wedge catheter inserted over the guidewire into the PA. The introducer tip is then threaded into the PA, and the wedge catheter is removed. The IJV is then serially dilated and the coaxial cannula 10 is threaded along the introducer into the IJV, through the right ventricle, and into the PA. The distal tip 70 of the first infusion tube 12 is sufficiently flexible as to allow it to be easily flexed about the longitudinal axis of the first elongate body 28 to navigate the IJV, right ventricle, and PA. The coaxial cannula 10 may include insertion depth markers and radiopaque markers for aiding the user in placing the coaxial cannula 10 in the right atrium. Once the position of the coaxial cannula 10 is acceptable, the introducer may be removed and the coaxial cannula 10 may clamped in place. For example, the coaxial cannula 10 may be secured to the patient's neck using a suture. FIG. 12 shows the coaxial cannula 10 positioned in the patient according to some embodiments of the disclosure. In particular, the second distal end 44 is positioned at least within proximity with the right atrium, while the first distal end 32 and the distal tip 70 extend through the main pulmonary artery and at least into proximity with either the left or right branch of the pulmonary branch of the pulmonary artery. The coaxial cannula 10 is inserted into the patient such that the infusion apertures 18 discharge blood into the main pulmonary artery. Additionally, the distal tip 70 may be provided with tip infusion apertures 79 to direct additional blood into the left and right branches of the pulmonary artery.

In the embodiment of the coaxial cannula 10 shown and described with reference to FIG. 4E, initial contact of the flap 90 with the distal end of the introducer causes the flap 90 to deflect to the opened position, permitting threading of the coaxial cannula 10 along the introducer. Removal of the introducer permits the flap 90 to bias to the closed position.

FIG. 13 illustrates the coaxial cannula 10 according to the embodiments of FIGS. 4F-4H positioned in the patient. Similarly to FIG. 12, the second distal end 44 is positioned at least within proximity with the right atrium, while the first distal end 32 and the distal tip 70 extend through the main pulmonary artery and at least into proximity with either the left or right branch of the pulmonary branch of the pulmonary artery. The distal tip 70 is curved to better position and retain the distal tip 70 within the pulmonary artery, decreasing patient discomfort and risk of vessel wall damage. In the case of the embodiment of the coaxial cannula 10 according to FIGS. 4F-4G, in which a shape memory alloy basket 80 is utilized with the distal tip 70, the distal tip 70 transitions to the curved profile shown in FIG. 13 as the temperature of the coaxial cannula 10 rises to match the patient's body temperature.

While several embodiments of a coaxial cannula are shown in the accompanying figures and described hereinabove in detail, other embodiments will be apparent to, and readily made by, those skilled in the art without departing from the scope and spirit of the invention. For example, it is to be understood that this disclosure contemplates, to the extent possible, that one or more features of any embodiment can be combined with one or more features of any other embodiment. Accordingly, the foregoing description is intended to be illustrative rather than restrictive.

What is claimed is:

1. A dual lumen coaxial cannula assembly comprising:
a first infusion tube having a first elongate body defining a first lumen therethrough, the first infusion tube having a proximal end, a distal end, and a sidewall extending circumferentially therebetween;
a second drainage tube co-axially aligned with the first infusion tube and having a second elongate body with a second lumen defined by a space between the first infusion tube and the second drainage tube, the second drainage tube having a proximal end, a distal end, and a sidewall extending circumferentially therebetween;
a connector attached to the proximal end of the first infusion tube and the proximal end on the second drainage tube; and
a distal tip defining a distal lumen therethrough, the distal tip having a proximal end, a distal end, and a sidewall extending circumferentially therebetween, the distal tip connected to the distal end of the first elongate body, wherein the distal tip is comprised of a material having a lower hardness than a hardness of the first elongate body.

2. The dual lumen coaxial cannula assembly of claim 1, wherein the distal tip is connected to a distal end of the first elongate body along a plane perpendicular to a longitudinal axis of the first infusion tube.

3. The dual lumen coaxial cannula assembly of claim 1, wherein the distal tip and the distal end of the first elongate body are tapered to increase a surface area of a connection between the distal tip and the distal end of the first elongate body.

4. The dual lumen coaxial cannula assembly of claim 1, wherein the distal tip and the distal end of the first elongate body are roughened to increase a surface area of a connection between the distal tip and the distal end of the first elongate body.

5. The dual lumen coaxial cannula assembly of claim 1, wherein the sidewall of the first infusion tube defines a shoulder such that a portion of the sidewall of the first infusion tube extending distally from the shoulder has a smaller outer diameter than a portion of the sidewall of the first infusion tube extending proximally from the shoulder; and
wherein the sidewall of the distal tip defines a bore adapted to receive the portion of the sidewall of the first infusion tube extending distally from the shoulder, such that a portion of the sidewall of the distal tip overlaps the portion of the sidewall of the first infusion tube extending distally from the shoulder.

6. The dual lumen coaxial cannula assembly of claim 5, further comprising a basket disposed between the sidewall of the distal tip and the sidewall of the first infusion tube, the basket comprising a plurality of individual members arranged to prevent collapse of the distal lumen.

7. The dual lumen coaxial cannula assembly of claim 6, wherein the basket is comprised of a shape memory material defining a first profile at a first temperature and a second profile at a second temperature.

8. The dual lumen coaxial cannula assembly of claim 7, wherein the first profile is a straight tubular profile;
wherein the first temperature is a temperature at which the distal tip is connected to the distal end of the first elongate body during a manufacturing process of the dual lumen coaxial cannula assembly;
wherein the second profile is a tubular structure curving about an axis perpendicular to a longitudinal axis of the first elongate body;
wherein the second temperature is a human body temperature; and
wherein the basket transitions from the first profile to the second profile as a temperature of the dual lumen coaxial cannula assembly transitions from the first temperature to the second temperature.

9. The dual lumen coaxial cannula assembly of claim 7, wherein the second profile is adapted for positioning in a bend of a patient's pulmonary artery.

10. The dual lumen coaxial cannula assembly of claim 1, wherein a profile of the distal tip defines a tubular structure curving about an axis perpendicular to a longitudinal axis of the first elongate body.

11. The dual lumen coaxial cannula assembly of claim 10, wherein the profile of the distal tip is adapted for positioning in a bend of a patient's pulmonary artery.

12. The dual lumen coaxial cannula assembly of claim 1, wherein the distal end of the distal tip defines an end feature shaped to reduce the prevalence of edges of the distal tip in order to ease venous insertion of the dual lumen coaxial cannula assembly into the patient and reduce the risk of vessel wall injury.

13. The dual lumen coaxial cannula assembly of claim 12, wherein the end feature has a profile of at least one of a balloon, a hemisphere, an oval, and a parabola.

14. The dual lumen coaxial cannula assembly of claim 1, wherein the distal tip further comprises a flap movable between a closed position preventing fluid flow out of the distal lumen and an opened position permitting fluid flow out of the distal lumen; and
wherein, in the closed position of the flap, fluid flow into the distal lumen creates backpressure forcing fluid out of one or more infusion apertures in the first elongate body.

15. The dual lumen coaxial cannula assembly of claim 14, wherein the flap is biased in the closed position and movable to the opened position by application of a force to a distal side of the flap.

16. A method of inserting a cannula assembly into a vasculature of a patient, the method comprising:
inserting an introducer into the jugular vein of the patient;
maneuvering a distal end of the introducer through the right atrium and right ventricle of the heart of the patient and into the pulmonary artery of the patient, the introducer having a length such that a proximal end of the introducer extends out of the body of the patient when the distal end of the introducer is positioned in the pulmonary artery;
sliding a central lumen of the cannula assembly over the introducer and maneuvering the cannula assembly along the introducer such that a first distal end of the cannula assembly is positioned at least within proximity of the pulmonary artery and such that a second distal end of the cannula assembly is at least within proximity of the right atrium; and
removing the introducer from the patient through the central lumen of the cannula assembly,
wherein the cannula assembly comprises a distal tip connected to the first distal end of the cannula assembly, wherein the distal tip is comprised of a material having a lower hardness than a hardness of the first distal end.

17. The method of claim 16, wherein, as the central lumen of the cannula assembly is slid over the introducer, the introducer deflects a flap in the cannula assembly from a closed position for preventing fluid flow out of a distal end of the central lumen to an opened position; and
wherein removing the introducer allows the flap to deflect back to the closed position for preventing fluid flow out of a distal end of the central lumen.

18. The method of claim 16, wherein, after removal of the introducer, the distal tip of the cannula assembly transitions from a first profile defining a straight tubular structure to a second profile defining a tubular structure curved about an axis perpendicular to a longitudinal axis of the cannula assembly.

19. The method of claim 18, wherein the transition from the first profile to the second profile is caused by a temperature change of a shape memory material forming at least a portion of the distal tip.

20. The method of claim 19, wherein the temperature change comprises a change from a first temperature to a second temperature,
wherein the first temperature is a temperature at which the distal tip is connected to the distal end of the first elongate body during a manufacturing process of the cannula assembly, and
wherein the second temperature is a body temperature of the patient.
